(19) United States
(12) Patent Application Publication
(10) Pub. No.: US 2003/0175366 A1
(43) Pub. Date: Sep. 18, 2003

(54) Plant Extracts and Methods of Treating Skin Therewith

(76) Inventors: Gilles Pauly, Nancy (FR); Phillippe Moser, Essey-Les-Nancy (FR); Louis Danoux, Sauixures-Les-Nancy (FR)

Correspondence Address: COGNIS CORPORATION, 2500 RENAISSANCE BLVD., SUITE 200, GULPH MILLS, PA, 19406

(21) Appl. No.: 10/257,695

(22) PCT Filed: Feb. 3, 2001
(86) PCT No.: PCT/EP01/01171

(30) Foreign Application Priority Data
Apr. 14, 2000 (EP) 00440107.1

(51) Int. Cl. A61K 35/78; A61K 7/06

(52) U.S. Cl. 424/725; 424/74

(57) ABSTRACT

Disclosed is the use of natural substances from the group formed by plant extracts from the solananaceae, from amaranthaceae and monimiaceae family in the production of cosmetic preparations. The invention also relates to the use thereof as active ingredients in the production of an agent for increasing G6PDH activity in metabolism.

### FIELD OF THE INVENTION

The invention is in the fields of cosmetics and pharmacy and relates firstly to the use of certain plant extracts in the production of cosmetic preparations and secondly to the use of the extracts as active ingredients for increasing the activity of certain enzymes in metabolism, and also to a method of evaluating active ingredients against skin aging.

### PRIOR ART

Although skin aging has many causes, the active ingredients developed to combat it by the cosmetics industry can in principle be divided into two groups. On the one hand there are active ingredients which protect the cells against oxidative stress or proteolysis (protective effect), and on the other hand there are active ingredients which stimulate cell regeneration (healing effect). It is the latter which have gained in importance in recent years although their development requires an exact understanding of the biochemical processes in the skin. These active ingredients include, for example, enzymes, which counter damage by UV light or environmental toxins, but also those which promote the synthesis of essential cell constituents, such as, for example, structural proteins, membrane lipids or DNA. Thus, cosmetic preparations on the market already nowadays comprise substances, for example DNA fragments, which stimulate IL-6 or P53 activity, proteins for stimulating collagen synthesis or ribosomes of E. coli for stimulating the growth of keratinocytes.

However, the present patent application is based on another finding. Glucose-6-phosphate dehydrogenase (G6PDH) is an enzyme which catalyzes the first steps of the so-called “pentose pathway”. During the first step, the conversion of glucose-6-phosphate (G6P) into 6-phosphogluconate (6PG), G6PDH requires, as coenzyme, nicotinamide adenine dinucleotide phosphate (NADP), which is reduced to NADPH2. The reduced form of this coenzyme can catalyze a large number of enzymatic reactions, such as, for example, the recycling of glutathione or lipid synthesis. In addition, the last step of the pentose pathway produces the most important precursor for DNA, deoxyribose. According to the investigations by Weber and Korting in J. Invest. Dermatol. 42, 167-169 (1964), G6PDH and 6-phosphatogluconate dehydrogenase (6PGDH) are to be found just as much as a number of other glycolysis enzymes (e.g. aldolase) in the upper layers of the human skin. On the basis of a large number of further investigations, the applicant has found that G6DPH not only plays an important role in skin metabolism, but can also serve in vitro as a model system for the effectiveness of active ingredients against skin aging.

The object of the patent application was therefore to provide those active ingredients which stimulate G6PDH activity in the fibroblasts, as far as possible without having an adverse effect on the DNA content of the cells.

### DESCRIPTION OF THE INVENTION

The invention provides for the use of natural substances chosen from the group formed by extracts of plants from the Solanaceae family, the Amaranthaceae family and the Monimiaceae family in the production of cosmetic preparations.

The invention further provides for the use of natural substances chosen from the group formed by extracts of plants from the Solanaceae family, the Amaranthaceae family and the Monimiaceae family as active ingredients for the production of an agent for increasing the G6PDH activity in metabolism.

Surprisingly, it has been found that the extracts of said plants or the active principles present therein achieve the set object in an advantageous manner. Compared with the standard, the G6PDH activity could be increased by up to 140% without the DNA content being significantly reduced. The substances are thus best suited for the production of cosmetic preparations, specifically skin-treatment agents and in particular agents against skin aging.

Plant Extracts

Typical examples of plants whose extracts can be used for the purposes of the invention are plants of the genera Physalis, Achyranthes and/or Peumus.

In a particular embodiment, extracts of the plants of the genera Physalis, Achyranthes and/or Peumus are accordingly used according to the invention.

In a further particular embodiment, extracts of Physalis minima, Achyranthes bidentata, Achyranthes aspera and/or Peumus boldo are used according to the invention.

Physalia minima Linn, which belongs to the Solanaceae family, is an annual herb which reaches a height of 15 to 30 cm and can be found growing wild in India, Ceylon, Afghanistan and in the tropical zones of Africa and Australia. Its fruits have a diuretic effect, while leaves and roots are used for medicinal purposes, for example against snake bites and scorpion stings. The bittering substances and steroid components of the leaves have now largely been explained. The steroids are essentially physalin A, physalin B, physalin C, physalin D, 5β,6β-epoxyphysalin, dihydroxyphysalin B, whitaphysalin A, whitaphysalin B and whitaphysalin C. In addition, phenolic acids, chlorogenic acids and flavone derivatives, such as, for example, quercetin-3-O-galactoside have been found.

Achyranthes bidentata and Achyranthes aspera: These plants belong to the Amaranthaceae family and are annual plants which grow vertically to a height of 1 m which are indigenous to Asia, in particular Korea, China, Vietnam, Japan and India. The plants are used widely in traditional medicine and are used, for example, as a diuretic and antirheumatic, but also to counter dental pain or menstrual discomfort. The leaves and seeds of both plants are edible and are consumed particularly in India as a vegetable and the seeds are used as grain for baking. The known ingredients include saponines, steroids, sterols (sitosterol), polysaccharides, alkaloids, phenolic acids (caffeic acid), quinones and flavone derivatives (rutin, isoquercetin, astragalin).

Peumus boldus: The plant Peumus boldus belongs to the Monimiaceae family. From the leaves and bark it is possible to extract the aporphine alkaloid boldin, which constitutes the essential active constituent of these extracts and which can be used according to the invention as a natural substance.

In a particular embodiment, accordingly, extracts are used according to the invention which comprise active ingredients chosen from the group formed by carotenoids, flavone derivatives, phenolic acids, chlorogenic acids, steroids, aporphine alkaloids, sterols and terpenes.

According to the invention, preference is given to using the carotenoids, flavone derivatives, phenolic acids, chlorogenic acids, steroids, aporphine alkaloids, sterols and terpenes which can be extracted from the preferred plants of the genera Physali, Achyranthes and Peumus. For the plants of the genus Physalis, preferred active ingredients include the steroids physalin A, physalin B, physalin C, physalin D, 5β,662 -epoxyphysalin, dihydroxyphysalin B and the steroids whitaphysalin A, whitaphysalin B, whitaphysalin C. All preferred steroids are types of steroid lactones. Further preferred active ingredients from the genus Physalis are phenolic acids, such as chlorogenic acids and caffeic acid, carotenoids, and flavone derivatives, such as, for example, quercitin-3-O-galactoside.

For the plants of the genus Achyranthes, active ingredients preferred according to the invention include saponines, steroids, sterols, polysaccharides, alkaloids, phenolic acids (caffeic acid), quinones, terpenes and flavone derivatives, such as, for example, rutin, isoquercetin or astragalin. Particular preference is given to the use of sterols and terpenes from Achyranthes, in particular from Achyranthes bidentata. For the plants of the genus Peumus, active ingredients which are preferred according to the invention include the aporphine alkaloids, in particular boldin.

Natural substances which can be used for the purposes of the invention are the extracts of said plants, in particular extracts of the plants Physalis minima, Achyranthes bidentata, Achyranthes aspera and Peumus boldo, the active ingredients alone or as a mixture in combination of at least two active ingredients extracted from said plants and their preferred selection or the active ingredients alone or in combination of at least two active ingredients extracted from further plants or prepared synthetically.

The amount of natural substances or of extracts used can here be in the range from 0.001 to 5% by weight, preferably 0.005 to 1% by weight and in particular 0.01 to 0.5% by weight, based on the agents.

Extraction

The extracts can be prepared in a manner known per se, i.e. for example by aqueous, alcoholic or aqueous-alcoholic extraction from the plants or plant parts. With regard to suitable conventional extraction processes such as maceration, remaceration, digestion, agitation maceration, vortex extraction, ultrasound extraction, countercurrent extraction, percolation, repercolation, evacolation (extraction under reduced pressure), diacolation and solid-liquid extraction with continuous reflux, which is carried out in a Soxhlet extractor, which are known to the person skilled in the art and can all be used in principle, for the sake of simplicity, reference may be made, for example, to Hagers Handbuch der Pharmazeutischen Praxis [Hagers handbook of pharmaceutical practice], (5th Edition, Vol. 2, pp. 1026-1030, Springer Verlag, Berlin-Heidelberg-New York 1991). For industrial use, the percolation method is advantageous. Starting materials which can be used are fresh plants or plant parts, although the starting materials are usually dried plants and/or plant parts which can be mechanically comminuted prior to extraction. In this connection, all of the comminution methods known to the person skilled in the art are suitable, freeze grinding may be given as an example. Solvents which can be used for carrying out the extraction may be organic solvents, water (preferably hot water with a temperature of more than 80° C. and in particular of more than 95° C.) or mixtures of organic solvents and water, in particular low molecular weight alcohols with greater or lesser water contents. Particular preference is given to extraction with methanol, ethanol, pentane, hexane, heptane, acetone, propylene glycols, polyethylene glycols, and ethyl acetate, and mixtures thereof, and aqueous mixtures thereof. The extraction is usually carried out at 20 to 100° C., preferably at 30 to 90° C., in particular at 60 to 80° C. In a preferred embodiment, the extraction is carried out under an inert gas atmosphere to avoid oxidation of the active ingredients of the extract. This is of importance particularly for extractions at temperatures above 40° C. The extraction times are adjusted by the person skilled in the art depending on the starting material, the extraction method, the extraction temperature, the ratio of solvent to raw material inter alia. Following extraction, the resulting crude extracts can optionally be subjected to further customary steps, such as, for example, purification, concentration and/or decoloration. If desired, the extracts prepared in this way can, for example, be subjected to selective removal of individual undesired ingredients. In a preferred embodiment, the extracts are subjected to a fractionation. Particular preference is given to fractionation by liquid chromatography over synthetic resins based on ion exchanger resins or on adsorber resins, in particular over Amberlite XAD. The fractionation is preferably carried out with a stepwise gradient of the eluent. Preferred solvent mixtures which can be used with a stepwise gradient as eluent are water/methanol, water/ethanol, water/isopropanol and methanol/acetone. In the case of the aqueous mixtures, the starting mixture is preferably 20% by weight aqueous methanol and the mixture is changed in stages up to 100% by weight of methanol. This gives various fractions which can be divided according to the mixing ratio of the solvent mixture. Depending on the polarity of the solvent mixture, extract ingredients of varying polarity are eluted and fractionated. The extraction can be carried out to any desired degree of extraction, but is usually carried out to exhaustion. Typical yields (=amount of dry substance of the extract based on the amount of raw material used) during the extraction of dried leaves is in the range from 3 to 15% by weight, in particular 6 to 10% by weight. The present invention encompasses the finding that the extraction conditions and the yields of the end extracts can be chosen by the person skilled in the art in accordance with the desired field of use. These extracts, which generally have active substance contents (=solids contents) in the range from 0.5 to 10% by weight, can be used as they are, although it is likewise possible to remove the solvent by drying, in particular by spray-drying or freeze-drying. The extracts can also be used as starting materials for obtaining the abovementioned pure active ingredients, provided these cannot be prepared in a more simple and cost-effective manner by a synthetic route.

Consequently, the active contents in the extracts can be 5 to 100% by weight, preferably 50 to 95% by weight. The extracts themselves can be in the form of preparations which are aqueous and/or dissolved in organic solvents, and also in the form of spray- or freeze-dried solids. Suitable organic solvents in this connection are, for example, the aliphatic alcohols having 1 to 6 carbon atoms (e.g. ethanol), ketones (e.g. acetone), halogenated hydrocarbons (e.g. chloroform or methylene chloride), lower esters or polyols (e.g. glycerol or glycols).

In a preferred embodiment of the invention, said natural substances and/or extracts are used as agents against skin aging. Another term for this type of agent is also antiaging agent. These aging phenomena include, for example, any type of line and wrinkle formation.

The treatments include a slowing of aging processes in the skin. The increase in the G6PDH activity in metabolism leads to the use of the natural substances according to the invention as agents against skin aging. The aging phenomena may have the most diverse causes.

The invention further provides for the use of natural substances chosen from the group formed by carotenoids, in particular retinol and retinolic acid, flavone derivatives, phenolic acids, chlorogenic acids, steroids, aporphine alkaloids, sterols and terpenes as active ingredients in the production of an agent for increasing the G6PDH activity in metabolism. The natural substances can be extracted from other plants, or be prepared by a synthetic route.

The amount of natural substances used can be in the range from 0.001 to 5% by weight, preferably 0.005 to 1% by weight and in particular 0.01 to 0.5% by weight, based on the agents.

The present invention further provides a method of determining the effectiveness of an agent against skin aging in which the influence of the active ingredient on the activity of glucose-6-phosphate dehydrogenase in fibroblasts is determined.

Industrial Applicability

The extracts to be used according to the invention can serve for the production of cosmetic and/or pharmaceutical preparations, such as, for example, creams, gels, lotions, alcoholic and aqueous/alcoholic solutions, emulsions, wax/fatty compositions, stick preparations, powders or ointments. These compositions can also comprise, as further auxiliaries and additives, mild surfactants, oily substances, emulsifiers, perlescent waxes, bodying agents, thickeners, superfatting agents, stabilizers, polymers, silicone compounds, fats, waxes, lecithins, phospholipids, biogenic active ingredients, UV light protection factors, antioxidants, deodorants, antiperspirants, antidandruff agents, film formers, swelling agents, insect repellents, self-tanning agents, tyrosine inhibitors (depigmentation agents), hydrotropes, solubilizers, preservatives, perfume oils, dyes and the like.

Surfactants

Surface-active substances which may be present are anionic, nonionic, cationic and/or amphoteric or amphoteric surfactants, the content of which in the compositions is usually about 1 to 70% by weight, preferably 5 to 50% by weight and in particular 10 to 30% by weight. Typical examples of anionic surfactants are soaps, alkylbenzenesulfonates, alkanesulfonates, olefin sulfonates, alkyl ether sulfonates, glycerol ether sulfonates, α-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether)sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulfates, protein fatty acid condensates (in particular wheat-based vegetable products) and alkyl (ether) phosphates. If the anionic surfactants contain polyglycolether chains, these may have a conventional homologue distribution, but preferably have a narrowed homologue distribution.

Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucoronic acid derivatives, fatty acid N-alkylglucamides, protein hydrolysates (in particular vegetable products based on wheat), polyol fatty acid esters, sugar esters, sorbitan esters, polysorbates and amine oxides. If the nonionic surfactants contain polyglycol ether chains, these may have a conventional homologue distribution, but preferably have a narrowed homologue distribution. Typical examples of cationic surfactants are quaternary ammonium compounds, such as, for example, dimethyldistearylammonium chloride, and ester quats, in particular quaternized fatty acid trialkanolamine ester salts. Typical examples of amphoteric or zwitterionic surfactants are alkylbetains, alkylamidobetains, aminopropionates, aminoglycinates, imidazoliniumbetains and sulfobetains. Said surfactants are exclusively known compounds. With regard to structure and preparation of these substances, reference may be made to relevant review works, for example, J. Falbe (ed.), “Surfactants in Consumer Products”, Springer Verlag, Berlin, 1987, pp. 54-124 or J. Falbe (ed.), “Katalysatoren, Tenside und Mineralöladditive [Catalysts, surfactants and mineral oil additives]”, Thieme Verlag, Stuttgart, 1978, pp. 123-217. Typical examples of particularly suitable mild, i.e. particularly skin-compatible surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetains, amphoacetales and/or protein fatty acid condensates, the latter preferably based on wheat proteins.

Oily Substances

Suitable oily substances are, for example, Guerbet alcohols based on fatty alcohols having 6 to 18, preferably 8 to 10, carbon atoms, esters of linear C₆-C₂₂-fatty acids with linear or branched C₆-C₂₂-fatty alcohols and/or esters of branched C₆-C₁₃-carboxylic acids with linear or branched C₆-C₂₂-fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂-fatty acids with branched alcohols, in particular 2-ethylhexanol, esters of C₁₈-C₃₈-alkyl hydroxycarboxylic acids with linear or branched C₆-C₂₂-fatty alcohols (cf. DE 19756377 A1), in particular dioctyl malates, esters of linear and/or branched fatty acids with polyhydric alcohols (such as, for example, propylene glycol, dimerdiol or trimertriol) and/or Guerbet alcohols, triglycerides based on C₆-C₁₀-fatty acids, liquid mono-/di-/triglyceride mixtures based on C₆-C₁₈-fatty acids, esters of C₆-C₂₂-fatty alcohols and/or Guerbet alcohols with aromatic carboxylic acids, in particular benzoic acid, esters of C₂-C₁₂-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, such as, for example, dicaprylyl carbonate (Cetiol® CC), Guerbet carbonates based on fatty alcohols having 6 to 18, preferably 8 to 10, carbon atoms, esters of benzoic acid with linear and/or branched C₆-C₂₂-alcohols (e.g. Finsolv® TN), linear or branched, symmetrical or unsymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicon methicone types, inter alia) and/or aliphatic or naphthenic hydrocarbons, such as, for example, such as squalane, squalene or dialkylcyclohexanes.

Emulsifiers

Suitable emulsifiers are, for example, nonionogenic surfactants from at least one of the following groups:

addition products of from 2 to 30 mol of ethylene oxide and/or 0 to 5 mol of propylene oxide to linear fatty alcohols having 8 to 22 carbon atoms, to fatty acids having 12 to 22 carbon atoms, to alkylphenols having 8 to 15 carbon atoms in the alkyl group, and alkylamines having 8 to 22 carbon atoms in the alkyl radical;

alkyl and/or alkenyl oligoglycosides having 8 to 22 carbon atoms in the alk(en)yl radical and the ethoxylated analogs thereof;

addition products of from 1 to 15 mol of ethylene oxide to castor oil and/or hydrogenated castor oil;

addition products of from 15 to 60 mol of ethylene oxide to castor oil and/or hydrogenated castor oil;

partial esters of glycerol and/or sorbitan with unsaturated, linear or saturated, branched fatty acids having 12 to 22 carbon atoms and/or hydroxycarboxylic acids having 3 to 18 carbon atoms, and the adducts thereof with 1 to 30 mol of ethylene oxide;

partial esters of polyglycerol (average degree of self-condensation 2 to 8), polyethylene glycol (molecular weight 400 to 5 000), trimethylolpropane, pentaerythritol, sugar alcohols (e.g. sorbitol), alkyl glucosides (e.g. methyl glucoside, butyl glucoside, lauryl glucoside), and polyglucosides (e.g. cellulose) with saturated and/or unsaturated, linear or branched fatty acids having 12 to 22 carbon atoms and/or hydroxycarboxylic acids having 3 to 18 carbon atoms, and the adducts thereof with 1 to 30 mol of ethylene oxide;

mixed esters of pentaerythritol, fatty acids, citric acid and fatty alcohol as in German Patent 1165574 and/or mixed esters of fatty acids having 6 to 22 carbon atoms, methylglucose and polyols, preferably glycerol or polyglycerol,

mono-, di- and trialkyl phosphates, and mono-, di- and/or tri-PEG alkyl phosphates and salts thereof;

wool wax alcohols;

polysiloxane-polyalkyl-polyether copolymers and corresponding derivatives;

block copolymers, e.g. polyethylene glycol-30 dipolyhydroxystearates;

polymer emulsifiers, e.g. Pemulen grades (TR-1, TR-2) from Goodrich;

polyalkylene glycols, and

glycerol carbonate.

The addition products of ethylene oxide and/or of propylene oxide to fatty alcohols, fatty acids, alkylphenols or to castor oil are known, commercially available products. These are homologue mixtures whose average degree of alkoxylation corresponds to the ratio of the amounts of substance of ethylene oxide and/or propylene oxide and substrate with which the addition reaction is carried out. C_{12/18}-fatty acid mono- and diesters of addition products of ethylene oxide to glycerol are known from German Patent 2024051 as refatting agents for cosmetic preparations.

Alkyl and/or alkenyl oligoglycosides, their preparation and their use are known from the prior art. They are prepared, in particular, by reacting glucose or oligosaccharides with primary alcohols having 8 to 18 carbon atoms. With regard to the glycoside radical, both monoglycosides, in which a cyclic sugar radical is glycosidically bonded to the fatty alcohol, and also oligomeric glycosides having a degree of oligomerization of up to, preferably, about 8, are suitable. The degree of oligomerization here is a statistical average value which is based on a homologue distribution customary for such technical-grade products.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid moglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride, and the technical-grade mixtures thereof which may also comprise small amounts of triglyceride as a minor product of the preparation process. Likewise suitable are addition products of 1 to 30 mol, preferably 5 to 10 mol, of ethylene oxide to said partial glycerides.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate, and technical-grade mixtures thereof. Likewise suitable are addition products of from 1 to 30 mol, preferably 5 to 10 mol, of ethylene oxide to said sorbitan esters.

Typical examples of suitable polyglycerol esters are polyglyceryl-2 dipolyhydroxystearate (Dehymuls® PGPH), polyglycerol-3 diisostearate (Lameform® TGI), polyglyceryl-4 isostearate (Isolan® GI 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Bellina®), polyglyceryl-4 caprate (Polyglycerol Caprate T2010/90), polyglyceryl-3 cetyl ether (Chimexane® NL), polyglyceryl-3 distearate (Cremophor® GS 32) and polyglyceryl polyricinoleate (Admul® WOL 1403), polyglyceryl dimerate isostearate, and mixtures thereof. Examples of further suitable polyol esters are the mono-, di- and triesters, optionally reacted with 1 to 30 mol of ethylene oxide, of trimethylolpropane or pentaerythritol with lauric acid, coconut fatty acid, tallow fatty acid, palmitic acid, stearic acid, oleic acid, behenic acid and the like.

Furthermore, zwitterionic surfactants can be used as emulsifiers. The term “zwitterionic surfactants” refers to those surface-active compounds which carry at least one quaternary ammonium group and at least one carboxylate and one sulfonate group in the molecule. Particularly suitable zwitterionic surfactants are the so-called betaines, such as N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 carbon atoms in the alkyl or acyl group, and cocoacylaminoethylhydroxyethylcarboxymethyl glycinate. Particular preference is given to the fatty acid amide derivative known under the CTFA name Cocamidopropyl Betaine. Likewise suitable emulsifiers are ampholytic surfactants. The term “ampholytic surfactants” means those surface-active compounds which, apart from a C_{8/18}-alkyl or -acyl group in the molecule, contain at least one free amino group and at least one —COOH or —SO₃H group and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group. Particularly preferred ampholytic surfactants are N-cocoalkylaminopropionate, cocoacylaminoethylaminopropionate and C_{12/18}-acylsarcosine. Finally, cationic surfactants are also suitable emulsifiers, those of the ester quat type, preferably methyl-quaternized difatty acid triethanolamine ester salts, being particularly preferred.

Fats and Waxes

Typical examples of fats are glycerides, i.e. solid or liquid vegetable or animal products which consist essentially of mixed glycerol esters of higher fatty acids, suitable waxes are inter alia natural waxes, such as, for example, candelilla wax, carnauba wax, japan wax, esparto grass wax, cork wax, guaruma wax, rice germ oil wax, sugarcane wax, ouricury wax, montan wax, beeswax, shellac wax, spermaceti, lanolin (wool wax), uropygial grease, ceresin, ozokerite (earth wax), petrolatum, paraffin waxes, microcrystalline waxes; chemically modified waxes (hard waxes), such as, for example, montan ester waxes, sasol waxes, hydrogenated jojoba waxes, and synthetic waxes, such as, for example, polyalkylene waxes and polyethylene glycol waxes. In addition to the fats, suitable additives are also fat-like substances, such as lecithins and phospholipids. The term “lecithins” is understood by the person skilled in the art as meaning those glycerophospholipids which form from fatty acids, glycerol, phosphoric acid and choline by esterification. Lecithins are thus in the specialist field also often referred to as phosphatidylcholines (PC). Examples of natural lecithins which may be mentioned are the kephalines, which are also referred to as phosphatidic acids and represent derivatives of 1,2-diacyl-sn-glycerol-3-phosphoric acids. By contrast, the term “phospholipids” usually means mono- and, preferably, diesters of phosphoric acid with glycerol (glycerol phosphates) which are generally considered to be fats. In addition, sphingosines and sphingolipids are also suitable.

Pearlescent Waxes

Examples of suitable pearlescent waxes are: alkylene glycol esters, specifically ethylene glycol distearate; fatty acid alkanolamides, specifically coconut fatty acid diethanolamide; partial glycerides, specifically stearic acid monoglyceride; esters of polybasic, optionally hydroxy-substituted carboxylic acids with fatty alcohols having 6 to 22 carbon atoms, specifically long-chain esters of tartaric acid; fatty substances, such as, for example, fatty alcohols, fatty ketones, fatty aldehydes, fatty ethers and fatty carbonates, which have a total of at least 24 carbon atoms, specifically laurone and distearyl ether; fatty acids, such as stearic acid, hydroxystearic acid or behenic acid, ring-opening products of olefin epoxides having 12 to 22 carbon atoms with fatty alcohols having 12 to 22 carbon atoms and/or polyols having 2 to 15 carbon atoms and 2 to 10 hydroxyl groups, and mixtures thereof.

Bodying Agents and Thickeners

Suitable bodying agents are primarily fatty alcohols or hydroxy fatty alcohols having 12 to 22, and preferably 16 to 18, carbon atoms, and also partial glycerides, fatty acids or hydroxy fatty acids. Preference is given to a combination of these substances with alkyl oligoglucosides and/or fatty acid N-methylglucamides of identical chain length and/or polyglycerol poly-12-hydroxystearates. Suitable thickeners are, for example, Aerosil grades (hydrophilic silicas), polysaccharides, in particular xanthan gum, guar guar, agar agar, alginates and Tyloses, carboxymethylcellulose and hydroxyethylcellulose, and also relatively high molecular weight polyethylene glycol mono- and diesters of fatty acids, polyacrylates (e.g. Carbopols® and Pemulen grades from Goodrich; Synthalens® from Sigma; Keltrol grades from Kelco; Sepigel grades from Seppic; Salcare grades from Allied Colloids), polyacrylamides, polymers, polyvinyl alcohol and polyvinylpyrrolidone, surfactants, such as, for example, ethoxylated fatty acid glycerides, esters of fatty acids with polyols such as, for example, pentaerythritol or trimethylolpropane, fatty alcohol ethoxylates having a narrowed homologue distribution or alkyl oligoglucosides, and electrolytes such as sodium chloride and ammonium chloride.

Superfatting Agents

Superfatting agents which can be used are substances such as, for example, lanolin and lecithin, and polyethoxylated or acylated lanolin and lecithin derivatives, polyol fatty acid esters, monoglycerides and fatty acid alkanolamides, the latter also serving as foam stabilizers.

Stabilizers

Stabilizers which can be used are metal salts of fatty acids, such as, for example, magnesium, aluminum and/or zinc stearate or ricinoleate.

Polymers

Suitable cationic polymers are, for example, cationic cellulose derivatives, such as, for example, a quaternized hydroxyethylcellulose obtainable under the name Polymer JR 400® from Amerchol, cationic starch, copolymers of diallylammonium salts and acrylamides, quaternized vinylpyrrolidone-vinylimidazole polymers, such as, for example, Luviquat (BASF), condensation products of polyglycols and amines, quaternized collagen polypeptides, such as, for example, lauryldimonium hydroxypropyl hydrolyzed collagen (Lamequat® L/Grünau), quaternized wheat polypeptides, polyethyleneimine, cationic silicone polymers, such as, for example, amidomethicones, copolymers of adipic acid and dimethylaminohydroxypropyldiethylenetriamine (Cartaretins®/Sandoz), copolymers of acrylic acid with dimethyldiallylammonium chloride (Merquat® 550/ Chemviron), polyaminopolyamides, as described, for example, in FR 2252840 A, and crosslinked water-soluble polymers thereof, cationic chitin derivatives, such as, for example, quaternized chitosan, optionally in microcrystalline dispersion, condensation products from dihaloalkyls, such as, for example, dibromobutane with bisdialkylamines, such as, for example, bis-dimethylamino-1,3-propane, cationic guar gum, such as, for example, Jaguar® CBS, Jaguar® C-17, Jaguar® C-16 from Celanese, quaternized ammonium salt polymers, such as, for example, Mirapol® A-15, Mirapol® AD-1, Mirapol® AZ-1 from Miranol.

Suitable anionic, zwitterionic, amphoteric and nonionic polymers are, for example, vinyl acetate-crotonic acid copolymers, vinylpyrrolidone-vinyl acrylate copolymers, vinyl acetate-butyl maleate-isobornyl acrylate copolymers, methyl vinyl ether-maleic anhydride copolymers and esters thereof, uncrosslinked polyacrylic acids and polyacrylic acids crosslinked with polyols, acrylamidopropyltrimethylammonium chloride-acrylate copolymers, octylacrylamide-methyl methacrylate-tert-butylaminoethyl methacrylate-2-hydroxypropyl methacrylate copolymers, polyvinylpyrrolidone, vinylpyrrolidone-vinyl acetate copolymers, vinylpyrrolidone-dimethylaminoethyl methacrylate-vinylcaprolactam terpolymers, and optionally derivatized cellulose ethers and silicones. Further suitable polymers and thickeners are listed in Cosm. Toil. 108, 95 (1993).

Silicone Compounds

Suitable silicone compounds are, for example, dimethylpolysiloxanes, methylphenylpolysiloxanes, cyclic silicones, and amino-, fatty-acid-, alcohol-, polyether-, epoxy-, fluorine-, glycoside- and/or alkyl-modified silicone compounds, which can either be liquid or in resin form at room temperature. Also suitable are simethicones, which are mixtures of dimethicones having an average chain length of from 200 to 300 dimethylsiloxane units and hydrogenated silicates. A detailed review of suitable volatile silicones can additionally be found in Todd et al., Cosm. Toil. 91, 27 (1976).

UV Light Protection Filters and Antioxidants

UV light protection factors are, for example, to be understood as meaning organic substances (light protection filters) which are liquid or crystalline at room temperature and which are able to absorb ultraviolet rays and give off the absorbed energy again in the form of longer-wavelength radiation, e.g. heat. UVB filters can be oil-soluble or water-soluble. Examples of oil-soluble substances are:

3-benzylidenecamphor or 3-benzylidenenorcamphor and derivatives thereof, e.g. 3-(4-methylbenzylidene)-camphor, as described in EP 0693471 B1;

4-aminobenzoic acid derivatives, preferably 2-ethylhexyl 4-(dimethylamino)benzoate, 2-octyl 4-(dimethylamino)benzoate and amyl 4-(dimethylamino)-benzoate;

esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di-2-ethylhexyl 4-methoxybenzmalonate [sic];

triazine derivatives, such as, for example, 2,4,6-trianilino(p-carbo-2′-ethyl-1′-hexyloxy)-1,3,5-triazine and octyltriazone, as described in EP 0818450 A1 or dioctylbutamidotriazone (Uvasorb® HEB);

propane-1,3-diones, such as, for example, 1-(4-tert-butylphenyl)-3-(4′-methoxyphenyl)propane-1,3-dione;

ketotricyclo(5.2.1.0)decane derivatives, as described in EP 0694521 B1.

Suitable water-soluble substances are:

2-phenylbenzimidazole-5-sulfonic acid and the alkali metal, alkaline earth metal, ammonium, alkylammonium, alkanolammonium and glucammonium salts thereof;

sulfonic acid derivatives of benzophenones, preferably 2-hydroxy-4-methoxybenzophenone-5-sulfonic acid and its salts;

sulfonic acid derivatives of 3-benzylidenecamphor, such as, for example, 4-(2-oxo-3-bornylidenemethyl)-benzenesulfonic acid and 2-methyl-5-(2-oxo-3-bornylidene)sulfonic acid and salts thereof.

Suitable typical UV-A filters are, in particular, derivatives of benzoylmethane, such as, for example, 1-(4′-tert-butylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds, as described in DE 19712033 A1 (BASF). The UV-A and UV-B filters can of course also be used in mixtures. Particularly favorable compositions consist of the derivatives of benzoylmethane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid, preferably 2-ethylhexyl 4-methoxycinnamate and/or propyl 4-methoxycinnamate and/or isoamyl 4-methoxycinnamate. Advantageously, such combinations are combined with water-soluble filters such as, for example, 2-phenylbenzimidazole-5-sulfonic acid and their alkali metal, alkaline earth metal, ammonium, alkylammonium, alkanolammonium and glucammonium salts.

As well as said soluble substances, insoluble light protection pigments, namely finely dispersed metal oxides or salts, are also suitable for this purpose. Examples of suitable metal oxides are, in particular, zinc oxide and titanium oxide and also oxides of iron, zirconium, silicon, manganese, aluminum and cerium, and mixtures thereof. Salts which may be used are silicates (talc), barium sulfate or zinc stearate. The oxides and salts are used in the form of the pigments for skincare and skin-protective emulsions and decorative cosmetics. The particles here should have an average diameter of less than 100 nm, preferably between 5 and 50 nm and in particular between 15 and 30 nm. They can have a spherical shape, but it is also possible to use particles which have an ellipsoidal shape or a shape deviating in some other way from the spherical form. The pigments can also be surface-treated, i.e. hydrophilicized or hydrophobicized. Typical examples are coated titanium dioxides, such as, for example, titanium dioxide T 805 (Degussa) or Eusolex® T2000 (Merck). Suitable hydrophobic coating agents are here primarily silicones and, specifically in this case, trialkoxyoctylsilanes or simethicones. In sunscreens, preference is given to using so-called micro- or nanopigments. Preference is given to using micronized zinc oxide. Further suitable UV light protection filters are given in the review by P. Finkel in SÖFW-Journal 122, 543 (1996) and Parf. Kosm. 3, 11 (1999).

As well as the two abovementioned groups of primary light protection substances, it is also possible to use secondary light protection agents of the antioxidant type; these interrupt the photochemical reaction chain which is triggered when UV radiation penetrates the skin. Typical examples thereof are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of gum benzoin, rutic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, super-oxide dismutase, zinc and derivatives thereof (e.g. ZnO, ZnSO₄) selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active ingredients which are suitable according to the invention.

Biogenic Active Ingredients

Biogenic active ingredients are to be understood as meaning, for example, tocopherol, tocopherol acetate, tocopherol palmitate, ascorbic acid, (deoxy)ribonucleic acid and fragmentation products thereof, retinol, bisabolol, allantoin, phytantriol, panthenol, AHA acids, amino acids, ceramides, pseudoceramides, essential oils, plant extracts and vitamin complexes.

Deodorants and Antimicrobial Agents

Cosmetic deodorants counteract, mask or remove body odors. Body odors arise as a result of the effect of skin bacteria on apocrine perspiration, with the formation of degradation products which have an unpleasant odor. Accordingly, deodorants comprise active ingredients which act as antimicrobial agents, enzyme inhibitors, odor absorbers or odor masking agents. Suitable antimicrobial agents are, in principle, all substances effective against gram-positive bacteria, such as, for example, 4-hydroxybenzoic acid and its salts and esters, N-(4-chlorophenyl)-N′-(3,4-dichlorophenyl)urea, 2,4,4′-trichloro-2′-hydroxydiphenyl ether (triclosan), 4-chloro-3,5-dimethylphenol, 2,2′-methylenebis(6-bromo-4-chlorophenol), 3-methyl-4-(1-methylethyl)phenol, 2-benzyl-4-chlorophenol, 3-(4-chlorophenoxy)-1,2-propanediol, 3-iodo-2-propynyl butylcarbamate, chlorohexidine, 3,4,4′-trichlorocarbanilide (TTC), antibacterial fragrances, thymol, thyme oil, eugenol, oil of cloves, menthol, mint oil, farnesol, phenoxyethanol, glycerol monocaprate, glycerol monocaprylate, glycerol monolaurate (GML), diglycerol monocaprate (DMC), salicylic acid N-alkylamides, such as, for example, n-octylsalicylamide or n-decylsalicylamide.

Suitable enzyme inhibitors are, for example, esterase inhibitors. These are preferably trialkyl citrates, such as trimethyl citrate, tripropyl citrate, triisopropyl citrate, tributyl citrate and, in particular, triethyl citrate (Hydagen® CAT). The substances inhibit enzyme activity, thereby reducing the formation of odor. Other substances which are suitable esterase inhibitors are sterol sulfates or phosphates, such as, for example, lanosterol, cholesterol, campesterol, stigmasterol and sitosterol sulfate or phosphate, dicarboxylic acids and esters thereof, such as, for example, glutaric acid, monoethyl glutarate, diethyl glutarate, adipic acid, monoethyl adipate, diethyl adipate, malonic acid and diethyl malonate, hydroxycarboxylic acids and esters thereof, such as, for example, citric acid, malic acid, tartaric acid or diethyl tartrate, and zinc glycinate.

Suitable odor absorbers are substances which are able to absorb and largely retain odor-forming compounds. They lower the partial pressure of the individual components, thus also reducing their rate of diffusion. It is important that in this process perfumes must remain unimpaired. Odor absorbers are not effective against bacteria. They comprise, for example, as main constituent, a complex zinc salt of ricinoleic acid or specific, largely odor-neutral fragrances which are known to the person skilled in the art as “fixatives”, such as, for example, extracts of labdanum or styrax or certain abietic acid derivatives. The odor masking agents are fragrances or perfume oils, which, in addition to their function as odor masking agents, give the deodorants their respective fragrance note. Perfume oils which may be mentioned are, for example, mixtures of natural and synthetic fragrances. Natural fragrances are extracts from flowers, stems and leaves, fruits, fruit peels, roots, woods, herbs and grasses, needles and branches, and resins and balsams. Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, p-tert-butylcyclohexyl acetate, linalyl acetate, phenylethyl acetate, linalyl benzoate, benzyl formate, allyl cyclohexylpropionate, styrallyl propionate and benzyl salicylate. The ethers include, for example, benzyl ethyl ether, and the aldehydes include, for example, the linear alkanals having 8 to 18 carbon atoms, citral, citronellal, citronellyloxyacetaldehyde, cyclamen aldehyde, hydroxycitronellal, lilial and bourgeonal, the ketones include, for example, the ionones and methyl cedryl ketone, the alcohols include anethol, citronellol, eugenol, isoeugenol, geraniol, linalool, phenylethyl alcohol and terpineol, and the hydrocarbons include mainly the terpenes and balsams. Preference is, however, given to using mixtures of different fragrances which together produce a pleasing fragrance note. Essential oils of relatively low volatility, which are mostly used as aroma components, are also suitable as perfume oils, e.g. sage oil, camomile oil, oil of cloves, melissa oil, mint oil, cinnamon leaf oil, linden flower oil, juniper berry oil, vetiver oil, olibanum oil, galbanum oil, labdanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Antiperspirants reduce the formation of perspiration by influencing the activity of the eccrine sweat glands, thus counteracting underarm wetness and body odor. Aqueous or anhydrous formulations of antiperspirants typically comprise the following ingredients:

astringent active ingredients,

oil components,

nonionic emulsifiers,

coemulsifiers,

bodying agents,

auxiliaries, such as, for example, thickeners or complexing agents and/or

nonaqueous solvents, such as, for example, ethanol, propylene glycol and/or glycerol.

Suitable astringent antiperspirant active ingredients are primarily salts of aluminum, zirconium or of zinc. Such suitable antihydrotic active ingredients are, for example, aluminum chloride, aluminum chlorohydrate, aluminum dichlorohydrate, aluminum sesquichlorohydrate and complex compounds thereof, e.g. with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, e.g. with amino acids, such as glycine. In addition, customary oil-soluble and water-soluble auxiliaries may be present in antiperspirants in relatively small amounts. Such oil-soluble auxiliaries may, for example, be:

anti-inflammatory, skin-protective or perfumed essential oils,

synthetic skin-protective active ingredients and/or

oil-soluble perfume oils.

Customary water-soluble additives are, for example, preservatives, water-soluble fragrances, pH regulators, e.g. buffer mixtures, water-soluble thickeners, e.g. water-soluble natural or synthetic polymers, such as, for example, xanthan gum, hydroxyethylcellulose, polyvinylpyrrolidone or high molecular weight polyethylene oxides.

Film Formers

Customary film formers are, for example, chitosan, microcrystalline chitosan, quaternized chitosan, polyvinylpyrrolidone, vinylpyrrolidone-vinyl acetate copolymers, polymers of the acrylic acid series, quaternary cellulose derivatives, collagen, hyaluronic acid and salts thereof, and similar compounds.

Antidandruff Active Ingredients

Suitable antidandruff active ingredients are pirocton olamin (1-hydroxy-4-methyl-6-(2,4,4-trimethylpentyl)-2-(1H)-pyridinone monoethanolamine salt), Baypival® (climbazole), Ketoconazole®, (4-acetyl-1-{-4-[2-(2,4-dichlorophenyl) r-2-(1H-imidazol-1-ylmethyl)-1,3-dioxylan-c-4-ylmethoxyphenyl}piperazine, ketoconazole, elubiol, selenium disulfide, sulfur colloidal, sulfur polyethylene glycol sorbitan monooleate, sulfur ricinole polyethoxylate, sulfur tar distillates, salicyclic acid (or in combination with hexachlorophene), undecylenic acid monoethanolamide sulfosuccinate Na salt, Lamepon® UD (protein undecylenic acid condensate), zinc pyrithione, aluminum pyrithione and magnesium pyrithione/dipyrithione magnesium sulfate.

Swelling Agents

The swelling agents for aqueous phases may be montmorillonites, clay mineral substances, Pemulen, and alkyl-modified Carbopol grades (Goodrich). Other suitable polymers and swelling agents are given in the review by R. Lochhead in Cosm. Toil. 108, 95 (1993).

Insect Repellents

Suitable insect repellents are N,N-diethyl-m-toluamide, 1,2-pentanediol or ethyl butylacetylaminopropionate.

Self-Tanning Agents and Depigmentation Agents

A suitable self-tanning agent is dihydroxyacetone. Suitable tyrosine inhibitors, which prevent the formation of melanin and are used in depigmentation agents, are, for example, arbutin, ferulic acid, kojic acid, coumaric acid and ascorbic acid (vitamin C).

Hydrotropes

To improve the flow behavior, hydrotropes, such as, for example, ethanol, isopropyl alcohol, or polyols, can also be used. Suitable polyols here preferably have 2 to 15 carbon atoms and at least two hydroxyl groups. The polyols can also contain further functional groups, in particular amino groups or be modified with nitrogen. Typical examples are

glycerol;

alkylene glycols, such as, for example, ethylene glycol, diethylene glycol, propylene glycol, butylene glycol, hexylene glycol, and polyethylene glycols with an average molecular weight of from 100 to 1000 daltons;

technical-grade oligoglycerol mixtures with a degree of self-condensation of from 1.5 to 10, such as, for example, technical-grade diglycerol mixtures with a diglycerol content of from 40 to 50% by weight;

methylol compounds, such as, for example, trimethylolethane, trimethylolpropane, trimethylolbutane, pentaerythritol and dipentaerythritol;

lower alkyl glucosides, in particular those with 1 to 8 carbon atoms in the alkyl radical, such as, for example, methyl and butyl glucoside;

sugar alcohols having 5 to 12 carbon atoms, such as, for example, sorbitol or mannitol,

sugars having 5 to 12 carbon atoms, such as, for example, glucose or sucrose;

amino sugars, such as, for example, glucamine;

dialcohol amines, such as diethanolamine or 2-amino-1,3-propanediol.

Preservatives

Suitable preservatives are, for example, phenoxy ethanol, formaldehyde solution, parabenes, pentanediol or sorbic acid, and the other classes of substance listed in Annex 6, Part A and B of the Cosmetics Directive.

Perfume Oils

Perfume oils which may be mentioned are mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamon, costus, iris, calmus), woods (pine wood, sandalwood, guaiac wood, cedarwood, rosewood), herbs and grasses (tarragon, lemon grass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexylpropionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamen aldehyde, hydroxycitronellal, lilial and bourgeonal, and the ketones include, for example, the ionones, α-isomethylionone and methyl cedryl ketone, the alcohols include anethole, citronellol, eugenol, isoeugenol, geraniol, linalool, phenylethyl alcohol and terpineol, and the hydrocarbons include predominantly the terpenes and balsams. Preference is, however, given to using mixtures of different fragrances which together produce a pleasing fragrance note. Essential oils of relatively low volatility, which are mostly used as aroma components, are also suitable as perfume oils, e.g. sage oil, camomile oil, oil of cloves, melissa oil, mint oil, cinnamon leaf oil, linden blossom oil, juniper berry oil, vetiver oil, olibanum oil, galbanum oil, labolanum oil and lavandin oil. Preference is given to using bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

Dyes

Dyes which can be used are the substances which are approved and suitable for cosmetic purposes, as are summarized, for example, in the publication “Kosmetische Färbemittel” [Cosmetic Colorants] from the Farbstoffkommission der Deutschen Forschungsgemeinschaft [Dyes Commission of the German Research Council], Verlag Chemie, Weinheim, 1984, pp. 81-106. These dyes are normally used in concentrations of from 0.001 to 0.1% by weight, based on the total mixture.

The total amount of auxiliaries and additives can be 1 to 50% by weight, preferably 5 to 40% by weight, based on the compositions. The compositions can be prepared by customary cold or hot processes; preference is given to using the phase-inversion temperature method.

### EXAMPLES

### Example 1

Preparation of an Extract from Physalis minima

In a glass reactor, 200 g of comminuted leaves of Physalis minima were dispersed in 2 l of 70% strength by weight aqueous methanol. The mixture was extracted over a period of 1 h under reflux and constant agitation, then cooled, filtered over a filter with a mesh width of 0.45 μm, the methanol was distilled at 30° C. under reduced pressure and the aqueous phase which remained was lyophilized. The yield, based on the leaves, was 15.8% by weight.

The extract was then subjected to a fractionation by liquid chromatography over Amberlite XAD 1180. Elution was carried out with a stepwise gradient from 20% by weight of aqueous methanol to 100% methanol. The extract without fractionation and three different fractions were then tested.

TABLE 1
Fractions from the extract of Physalis minima to be
tested

| Fraction | 1 | 2 | 3 |
|---|---|---|---|
| Eluent (% by weight methanol) | 40-60 | 60 | 60-80 |

### Example 2

Preparation of an Extract from Achyranthes bidentata

In a glass reactor, 300 g of comminuted leaves and stems of Achyranthes bidentata were dispersed in 3 1 of 96% strength by weight aqueous ethanol. The mixture was extracted over a period of 1 h with reflux and constant agitation, then cooled, and the solution was filtered over a filter with a mesh width of 0.45 μm. The residue which remained was extracted a second time under the same conditions. The filtrates were combined, treated with 1.5% by weight of activated carbon for decolorization and filtered once again. The resulting still slightly brownish filtrate was then freed from ethanol at 40° C. under reduced pressure and the aqueous phase was evaporated to an active substance content of 50% by weight. The yield was 7.6% by weight based on the feed material.

The extract was then subjected to a fractionation by liquid chromatography over Amberlite XAD 1180. The elution was carried out with a stepwise gradient from 20% by weight of aqueous methanol to 100% by weight of methanol. An extract without fractionation and the fraction with 100% by weight of methanol were then tested.

### Example 3

The G6PDH activity was determined in accordance with the microprocess described by Garidelli de Quincenet in Annual Dermatol. Venereol. 107(12), 1163-1170 (1980). The G6PDH activity was determined in accordance with the process described by Natsuko Okada and Yukio Kitano in: Arch. Dermatol. Res., 271(3): 341-346, 1981 by in vitro determination of the enzymatic activity of the glucose-6-phosphate dehydrogenase in human fibroblasts.

The DNA content was determined in accordance with the method described by Desaulniers in Toxic. In vitro 12(4), 409-422 (1998). The incubation time of the fibroblasts was in each case 3 days and 6 days. The results are summarized in Table 2. In each case, the average of 4 experiments with triple determination is given.

TABLE 2
G6PDH activity and DNA-data in rel-%

| | | DNA | G6PDH | DNA | G6PDH |
|---|---|---|---|---|---|
| | | content | activity | content | activity |
| | Conc. | after 3 | after 3 | after 6 | after 6 |
| Feed | % | days | days | days | days |
| material | by wt. | rel-% | rel-% | rel-% | rel-% |
| Control | 0 | 100 | 100 | 100 | 100 |
| Extract as in Example 1 | 0.002 | 98 | 115 | 83 | 208 |
| (without fractionation) | | | | | |
| Fraction 1 (Example 1) | 0.002 | 81 | 117 | 84 | 163 |
| Fraction 2 (Example 1) | 0.002 | 74 | 154 | 74 | 241 |
| Fraction 3 (Example 1) | 0.0003 | 95 | 154 | 91 | 173 |
| Extract as in Example 2 | 0.005 | 146 | 111 | 75 | 236 |
| (without fractionation) | | | | | |
| Fraction 1 (Example 2) | 0.0003 | 95 | 109 | 87 | 162 |
| Boldin index B3916 | 0.005 | 97 | 200 | — | — |
| (Sigma) | | | | | |
| Retinol | 0.005 | 95 | 400 | — | — |
| Retinoic acid | 0.00003 | 76 | 134 | 98 | 130 |

The results in Table 2 show that the extract of Physalis minima with a concentration of 0.002% by weight has increased the activity of G6PDH in human fibroblasts after 6 days extremely. The fractions 1 and 2 from the extracts of Physalis minima exhibit, at a concentration of 0.002% by weight after an incubation time of 6 days, an increased activity of the G6PDH, and fraction 2 exhibits an increase in activity after just 3 days. By comparison, fraction 3 shows, at a significantly lower concentration of only 0.0003% by weight, an increase in the desired activity after incubation for 3 and 6 days. With these results, it can be shown that the activity of G6PDH can be increased by extracts of the plant Physalis minima.

The extracts and the methanol fraction of the extract of the plant Achyranthes bidentata show an increase in the activity at a concentration of 0.005% by weight after incubation for 3 and 6 days. The fraction from the methanol fractionation is effective even at a concentration of 0.0003% by weight. The results can demonstrate that the extracts of the plant Achyranthes bidentata stimulate and can increase the activity of G6PDH.

TABLE 3
Cosmetic preparations (water, preservative ad 100% by
weight)

| Composition (INCI) | 1 | 2 | 3 | 4 | 5 | 6 | 7 | 8 | 9 | 10 |
|---|---|---|---|---|---|---|---|---|---|---|
| Emulgade ® SE | 5.0 | — | 5.0 | 4.0 | — | — | — | — | — | — |
| Glyceryl Sterate (and) | | | | | | | | | | |
| Ceteareth 12/20 (and) | | | | | | | | | | |
| Cetearyl Alcohol (and) | | | | | | | | | | |
| Cetyl Palmitate | | | | | | | | | | |
| Eumulgin ® B1 | — | — | — | 1.0 | — | 1.0 | — | — | — | — |
| Ceteareth-12 | | | | | | | | | | |
| Lameform ® TGI | — | — | — | — | — | — | 4.0 | — | 4.0 | — |
| Polyglyceryl-3 | | | | | | | | | | |
| Isostearate | | | | | | | | | | |
| Dehymuls ® PGPH | — | 5.0 | — | — | 5.0 | 4.0 | — | 4.0 | — | 4.0 |
| Polyglyceryl-2 | | | | | | | | | | |
| Dipolyhdroxystearate | | | | | | | | | | |
| Monomuls ® 90-O 18 | — | — | — | — | — | — | 2.0 | — | 2.0 | — |
| Glyceryl Oleate | | | | | | | | | | |
| Cetiol ® HE | — | 3.0 | — | — | — | — | — | 2.0 | — | 2.0 |
| PEG-7 Glyceryl Cocoate | | | | | | | | | | |
| Cetiol ® OE | — | 3.0 | — | — | 3.0 | 3.0 | 5.0 | 6.0 | 5.0 | 6.0 |
| Dicaprylyl Ether | | | | | | | | | | |
| Cetiol ® PGL | — | — | — | 3.0 | — | — | 9.0 | 9.0 | | |
| Hexyldecanol (and) | | | | | | | | | | |
| Hexyldecyl Laurate | | | | | | | | | | |
| Cetiol ® SN | 3.0 | — | 3.0 | — | — | — | — | — | 5.0 | 3.0 |
| Cetearyl Isononanoate | | | | | | | | | | |
| Cetiol ® V | 3.0 | — | 3.0 | — | 3.0 | — | — | — | 5.0 | 6.0 |
| Decyl Oleate | | | | | | | | | | |
| Myritol ® 318 | — | — | — | 3.0 | — | 3.0 | 5.0 | 5.0 | 5.0 | 5.0 |
| Coco Caprylate | | | | | | | | | | |
| Caprate | | | | | | | | | | |
| Beeswax | — | — | — | — | — | — | 7.0 | 5.0 | 7.0 | 5.0 |
| Nutrilan ® Elastin E20 | 2.0 | — | — | — | 2.0 | — | — | — | — | — |
| Hydrolyzed Elastin | | | | | | | | | | |
| Nutrilan ® I-50 | — | 2.0 | 2.0 | — | — | — | — | — | — | — |
| Hydrolyzed Collagen | | | | | | | | | | |
| Gluadin ® AGP | — | — | — | 0.5 | — | 0.5 | — | — | — | — |
| Hydrolyzed Wheat | | | | | | | | | | |
| Gluten | | | | | | | | | | |
| Gluadin ® WK | — | — | — | — | — | — | 0.5 | 0.5 | 0.5 | 0.5 |
| Sodium Cocoyl | | | | | | | | | | |
| Hydrolyzed Wheat | | | | | | | | | | |
| Protein | | | | | | | | | | |
| Extract as in Example | 1.0 | — | 1.0 | — | 1.0 | — | 1.0 | — | 1.0 | — |
| 1 | | | | | | | | | | |
| Extract as in Example | — | 1.0 | — | 1.0 | — | 1.0 | — | 1.0 | — | 1.0 |
| 2 | | | | | | | | | | |
| Hydagen ® CMF | 1.0 | 1.0 | 1.0 | 1.0 | 1.0 | 1.0 | 1.0 | 1.0 | 1.0 | 1.0 |
| Chitosan | | | | | | | | | | |
| Magnesium Sulfate | — | — | — | — | — | — | 1.0 | 1.0 | 1.0 | 1.0 |
| Heptahydrate | | | | | | | | | | |
| Glycerin (86% strength | 3.0 | | 3.0 | 5.0 | 3.0 | 5.0 | 5.0 | 3.0 | 5.0 | 3.0 |
| by weight) | | | | | | | | | | |

(1,2) soft cream, (3-6) moisturizing emulsion, (7-10) night cream

1. The use of natural substances chosen from the group formed by extracts of plants from the Solanaceae family the Amaranthaceae family and the Monimiaceae family in the production of cosmetic preparations.

2. The use of natural substances chosen from the group formed by extracts of plants from the Solanaceae family, the Amaranthaceae family and the Monimiaceae family as active ingredients for the production of an agent for increasing the G6PDH activity in metabolism.

3. The use as claimed in claims 1 and/or 2, characterized in that extracts of plants of the genera Physalis, Achyranthes and/or Peumus are used.

4. The use as claimed in at least one of claims 1 to 3, characterized in that extracts of Physalis minima, Achyranthes bidentata, Achyranthes aspera and/or Peumus boldo are used.

5. The use as claimed in at least one of claims 1 to 4, characterized in that extracts are used which comprise active ingredients chosen from the group formed by carotenoids, flavone derivatives, phenolic acids, chlorogenic acids, steroids, aporphine alkaloids, sterols and terpenes.

6. The use as claimed in at least one of claims 1 to 5, characterized in that the natural substances and/or the extracts are used in amounts of from 0.001 to 5% by weight, based on the agents.

7. The use as claimed in claim 6, characterized in that the active ingredient content in the extracts is 5 to 100% by weight.

8. The use as claimed in at least one of claims 1 to 7, characterized in that the natural substances are used as preparations which are aqueous and/or dissolved in organic solvents, or spray- or freeze-dried solids.

9. The use as claimed in claim 1 and/or 2 as agents against skin aging.

10. The use of natural substances chosen from the group formed by carotenoids, flavone derivatives, phenolic acids, chlorogenic acids, steroids, aporphine alkaloids, sterols and terpenes as active ingredients in the production of an agent for increasing the G6PDH activity in metabolism.

11. A method of determining the effectiveness of an agent against skin aging in which the influence of the active ingredient on the activity of glucose-6-phosphate dehydrogenase in fibroblasts is determined.
